(19) United States
(12) Patent Application Publication
(10) Pub. No.: US 2014/0249480 A1
(43) Pub. Date: Sep. 4, 2014

(54) Medicated Module with Automatic Activation Mechanism

(71) Applicants: David Richard Mercer, Warwickshire (GB); Garen Kouyoumjian, Warwickshire (GB); Malcolm Stanley Boyd, Warwickshire (GB)

(72) Inventors: David Richard Mercer, Warwickshire (GB); Garen Kouyoumjian, Warwickshire (GB); Malcolm Stanley Boyd, Warwickshire (GB)

(73) Assignee: SANOFI-AVENTIS DEUTSCHLAND GMBH, Frankfurt am Main (DE)

(21) Appl. No.: 14/112,057

(22) PCT Filed: Apr. 19, 2012
(86) PCT No.: PCT/EP12/57156

§ 371 Date: Oct. 16, 2013

(30) Foreign Application Priority Data
Apr. 21, 2011 (EP) 11163390.5

(51) Int. Cl. A61M 5/24; A61M 5/32

(52) U.S. Cl. CPC A61M 5/2448; A61M 5/326; A61M 5/3294; A61M 2005/1787
USPC 604/198

(57) ABSTRACT

Disclosed herein are various examples of a medicated module that can be attached to a drug delivery device such that a user can administer a user settable dose of a first medicament and a fixed dose of a second medicament through a single dispense interface. In one example, the medicated module includes (i) an outer housing having a hub that holds a first needle, (ii) a bypass housing including a reservoir containing a second medicament, (iii) a lower hub including a second needle, (iv) a slidable needle guard including at least one disengagement member, where the needle guard is configured to activate the medicated module by forcing the bypass housing to disengage from the outer housing at a pre-defined amount of proximal displacement of the needle guard; and (v) a biasing member, where the biasing member is configured to place the second needle in fluid communication with the first and second medicaments automatically after activation of the medicated module.

### CROSS REFERENCE TO RELATED APPLICATIONS

The present application is a U.S. National Phase Application pursuant to 35 U.S.C. §371 of International Application No. PCT/EP2012/057156 filed Apr. 19, 2012, which claims priority to European Patent Application No. 11163390.5 filed Apr. 21, 2011. The entire disclosure contents of these applications are herewith incorporated by reference into the present application.

### FIELD OF INVENTION

The present patent application relates to medical devices and methods for delivering multiple fluids and/or medicaments using a device having a single dose setting mechanism and a single dispense interface. The fluids and/or medicaments may be contained in one or more cartridges, reservoirs, containers or packages, each containing independent (single compound) or pre-mixed (co-formulated multiple compounds) drug agents. The disclosed device is of particular benefit where combination therapy is desirable, but not possible in a single formulation for reasons such as, but not limited to, stability, compromised therapeutic performance and toxicology.

### BACKGROUND

Certain disease states require and/or benefit from treatment using two or more different medicaments (i.e., combination therapy). For example, in some cases it might be beneficial to treat a diabetic with a long acting insulin and with a glucagon-like peptide-1 (GLP-1), which is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus.

Although certain disease states require and/or benefit from combination therapy, there are a number of potential problems associated with delivering two active medicaments or “drug agents” together. For instance, certain medicaments need to be delivered in a specific relationship with each other in order to deliver the optimum therapeutic dose. Additionally, the two active drug agents may interact with each other during the long-term shelf life storage of the formulation. Therefore, it may be advantageous to store the active drug agents separately and only combine them at the point of delivery, for example, by injection, needle-less injection, pumps, or inhalation. However, the process for combining the two active drug agents needs to be simple and convenient for the user to perform reliably, repeatedly, and safely.

A further problem that may arise with combination therapy is that the quantities and/or proportions of each active drug agent making up the combination therapy may need to be varied for each user and/or at different stages of each user's therapy. For example, one or more active drug agents may require a titration period to gradually introduce a patient to a “maintenance” dose. A further example would be if one active drug agent requires a non-adjustable fixed dose while the other is varied in response to a patient's symptoms and/or physical condition. Accordingly, certain pre-mixed formulations of multiple active drug agents may not be suitable as these pre-mixed formulations would have a fixed ratio of the active components, which could not be varied by the healthcare professional or user.

Additional problems may arise where combination therapy is required because many users cannot cope with having to use more than one drug delivery system or with having to make the necessary accurate calculation of the required combination dose. This is especially true for users with dexterity and/or computational difficulties.

In light of the above-mentioned problems, there exists a need to provide devices and methods for the delivery of two or more medicaments that require only a single dose setting step and a single injection step that is simple for the user to perform without complicated physical manipulations of the device. Further, there is a need to provide such a device that keeps the medicaments separated until the point of delivery.

### SUMMARY

Disclosed herein are various examples of a medicated module that can be attached to a drug delivery device (e.g., a pen injection device) such that a user can administer a user settable dose of a first medicament from the drug delivery device and a fixed dose of a second medicament from the medicated module (collectively “a combination dose”) through a single dispense interface, where only a single dose setting step is required, and where the medicaments are not combined until delivery. The medicated module transitions from a priming state to a delivery state automatically when the user (or someone assisting the user such as a physician) inserts the dispense interface into the user's skin such that the needle guard component of the medicated module retracts.

The combination dose may be predefined using a therapeutic dose profile. By defining the therapeutic relationship between the medicaments, the proposed system helps to ensure that a user receives the optimum therapeutic combination dose. This combination dose may be set and administered without the inherent risks that may be associated with multiple inputs, where the user is often called upon to calculate and set the correct dose combination each time that the device is used to administer a dose. The medicaments may each contain independent (single compound) or pre-mixed (co-formulated multiple compounds) drug agents. Although described as a first and second medicament, the medicaments may contain the same or substantially the same constituent parts. The medicaments can be fluids, defined herein as liquids, gases or powders that are capable of flowing and that change shape when acted upon by a force tending to change its shape. Alternatively, one of the medicaments may be a solid where such a solid may be carried, solubilized or otherwise dispensed with another fluid. In one example, a first medicament such as insulin may be contained within the drug delivery device and a second medicament such as a GLP-1 may be contained within the medicated module.

Although the present application specifically mentions insulin, insulin analogs or insulin derivatives, and GLP-1 or GLP-1 analogs as possible drug combinations, other drugs or drug combinations, such as an analgesics, hormones, beta agonists or corticosteroids, or a combination of any of the above-mentioned drugs can be used. Herein, the term “insulin” shall mean Insulin, insulin analogs, insulin derivatives or mixtures thereof, including human insulin or a human insulin analogs or derivatives. Examples of insulin analogs are, without limitation, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin or Des(B30) human insulin. Examples of insulin derivatives are, without limitation, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Herein the term “GLP-1” shall mean GLP-1, GLP-1 analogs, or mixtures thereof, including without limitation, exenatide (Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2), Exendin-3, Liraglutide, or AVE0010 (H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-ys-Lys-NH2).

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

One example of the medicated module includes (i) an outer housing having a proximal end, a distal end, and an outer surface, where the proximal end has a hub holding a first (or proximal) double-ended needle and a connector configured for attachment to a drug delivery device (e.g., a pen injection-type device) containing a first fluid or medicament, (ii) a bypass housing having a reservoir containing a second fluid or medicament, (iii) a lower hub that holds a second (or distal) double-ended needle (i.e., the dispense interface), (iv) a needle guard that can reduce the risk of accidental needle sticks before and after use and reduce the anxiety of users suffering from needle phobia, and (v) a biasing member such as a spring that is positioned between the lower hub and a proximally facing distal surface of the guard.

The needle guard may have a solid planar surface at its distal end that provides a large surface area that reduces the pressure exerted on the patient's skin, which allows the user to experience an apparent reduction in the force exerted against the skin. Preferably, the planar surface covers the entire distal end of the guard with the exception of a small needle pass through hole aligned axially with the needle. In one example, the pass through hole is no more than 10 times greater in diameter than the outer diameter of the second needle. The pass through hole diameter should be large enough for the user to see that the system is primed (i.e., see at least a drop of the first medicament being expelled through the distal end of the second needle) while not being so large that it is still possible to reach the end of the second needle with a finger. This difference between the diameter of the pass through hole and the needle also accommodates tolerances of various components of the medicated module. The needle guard may be configured to move axially in both the distal and proximal directions when pressed against and removed from an injection site of the user. When the second needle is removed or withdrawn from the patient, the guard is returned to a fully extended position. In some embodiments the medicated module may include features to lock out the guard after use.

In another example, the medicated module comprises (i) an outer housing including an upper hub located near a proximal end of the outer housing, where the upper hub holds a first needle, and where the proximal end is configured to attach to a drug delivery device, (ii) a bypass housing including a reservoir containing a second medicament, where the bypass housing is configured to be engaged with the outer housing prior to activation of the medicated module, (iii) a lower hub including a second needle, (iv) a slidable needle guard including at least one disengagement member, where the needle guard is configured to activate the medicated module by forcing the bypass housing to disengage from the outer housing at a pre-defined amount of proximal displacement of the needle guard and (v) a biasing member (e.g., a spring) located between a proximally facing internal surface of the needle guard and a distally facing external surface of the lower hub. The biasing member is configured to force the lower hub and the bypass housing in the proximal direction after activation of the medicated module, thereby placing the second needle in fluid communication with the first and second fluids.

The needle guard may be positioned such that the needle guard slides along an external surface of the outer housing or the needle guard may be positioned such that the needle guard slides along an internal surface of the outer housing. If the needle guard is positioned such that the needle guard slides along an external surface of the outer housing, the needle guard may include an opening for viewing indicia on an outer surface of the outer housing. Alternatively, the needle guard may comprise a transparent needle guard. Additionally, the indicia may indicate one or more of a priming state, an activated state, a delivery state, and a lock-out state.

The bypass housing and the outer housing are engaged prior to activation of the medicated module by engagement of respective engagement features. The bypass housing is prevented from moving in the proximal direction relative to the outer housing by compatible engagement features. The at least one engagement feature of the bypass housing may be a hook engagement feature. The at least one engagement feature of the outer housing may be a cutout engagement feature or a rib protrusion feature. A proximal surface of the at least one disengagement member and a distal surface of the at least one engagement feature on the bypass housing are each angled such that proximal displacement of the at least one disengagement member forces the bypass housing to rotate due to interaction of the angled surfaces. Such rotation continues until the bypass housing and the outer housing are disengaged, which allows the biasing member to force the lower hub and bypass housing proximally, thereby activating the medicated module.

The medicated module disclosed herein is configured to eliminate the need to have a user manually operate the medicated module to change the state of the module from a pre-activation/priming state (i.e., where only the first medicament from the drug delivery device can be delivered via the second needle) to an activated state (i.e., where both the first and second medicaments can be delivered via the second needle) and finally to a locked out state (i.e., where the device cannot be used once the medicament has been dispensed, typically defined as when the user has inserted the needle to the injection site and retracted it following dispense). Manually operated devices are sometimes not as intuitive as they could be and raise a potential risk of accidental misuse. The medicated module disclosed herein utilizes energy stored within the module that is present at least in part due to pre-compression of the spring. This stored energy is released during normal user operation of the module when the module is activated, which occurs when the outer housing is disengaged from the bypass housing and ultimately results in the second needle coming into fluid communication with both the first and second medicaments. The module is designed to make this activation imperceptible to the user, consequently making the user experience of the module very similar to that of a standard commercially available and accepted needle or safety needle (i.e. unpack module, attach to a drug delivery device, prime drug delivery device, insert into skin, inject a set dose along with single dose in the module). In this way, the module aims to reduce the risk of unintentional misuse and to improve usability by replicating an already accepted practice for similar injection methods. As the medicated module does not require the user to access external features on the module for the purposes of activation, the number of components and subsequent module size can be reduced/optimized.

Although principally described in this application as a single-use, potentially high-volume manufacture, and disposable device application, it should be understood that the medicated module may be reusable. For example, the medicated module may have a replaceable reservoir or may have an access port through which the second medicament can be refilled.

In operation, as the user presses the distal face of the needle guard against their skin the needle guard moves axially in the proximal direction. This axial motion of the guard causes a rotation of the bypass housing through the interaction of disengagement members of the guard and features of the bypass housing that are used to engage features of the outer housing when the medicated module is in a pre-activated state. In the pre-activated state or prior to activation the bypass housing is prevented from moving in the proximal direction relative to the outer housing. After a predefined amount of proximal axial displacement of the guard, the rotation of the bypass housing disengages the bypass housing from the outer housing. The spring is now free to release stored energy which forces the lower hub and the bypass housing in the proximal direction. This results in the first and second needles transitioning from a pre-activation/priming state to an activated state whereby a combination dose of the medicaments may be delivered. Further axial movement of the needle guard is required in order to pierce the user's skin and place the medicated module in a delivery state. This further retraction of the needle guard temporarily re-compresses the spring creating additional stored energy. Once the user removes the second needle from their skin and releases the force that they are applying to the system, the guard will return to its fully extended position.

A method is also provided for dispensing a combination dose using a medicated module described herein. First, the user attaches a medicated module containing a medicament to a drug delivery device containing another medicament. The user can then prime the system by setting and dispensing a small dose of the medicament in the drug delivery device. After priming, the user presses the needle guard against their skin which causes the module to automatically change from a pre-activation/priming state to an activated state where the dispense interface of the medicated module is in fluid communication with both medicaments. The user continues to press the guard against their skin until the guard can no longer move in the proximal direction relative to the outer housing. At this point the dispense needle has pierced the user's skin and the combination dose is ready for delivery. The user then activates the drug delivery device (e.g., actuates the delivery button of the device). Upon completion of the delivery procedure and removal of the dispense needle from the user's skin, the guard will return to its fully extended position. The medicated module may provide audible feedback to the user when the medicated module transitions from one state to another. For example, the medicated module may provide a mechanically generated click or perhaps an electronic indication when the medicated module is activated. This click could be generated utilizing the release of stored energy during device activation.

During dispense, substantially the entire amount of the second medicament in the medicated module is expelled as well as the selected or dialed dose of the first medicament. The reservoir may include a flow distributor to ensure that substantially all the single dose of second medicament is forced out of the reservoir by the first medicament during an injection. The flow distributor can be a separate stand alone insert or pin. Alternatively the flow distributor and the reservoir can be manufactured or assembled as a one-piece component where the flow distributor is integral with the reservoir. Such a unitary construction can be achieved utilizing, for example, design principles such as form fit, force fit or material fit, such as welding, gluing, or the like, or any combination thereof. The one-piece component may comprise one or more medicament flow channels. The reservoir and/or flow distributor can be constructed of any material that is compatible with the first and second medicaments. For example, COC (an amorphous polymer based on ethylene and norbonene, also referred to as cyclic olefin copolymer, ethylene copolymer, cyclic olefin polymer, or ethylene-norbornene copolymer); LCP (a liquid crystal polymer having an aramid chemical structure that includes linearly substituted aromatic rings linked by amide groups, and further can include partially crystalline aromatic polyesters based on p-hydroxybenzoic acid and related monomers and also highly aromatic polyesters); PBT (polybutylene terephthalate thermoplastic crystalline polymer or polyester); COP (a cyclic olefin polymer based on ring-opening polymerization of norbornene or norbornene-derivatives); HDPE (high density polyethylene); and SMMA (styrene methyl methacrylate copolymer based on methyl methacrylate and styrene). A preferred material is one that is typically used to manufacture septa or pistons (bungs) found in multi-dose medicament cartridges, however, any other material that is compatible with the drug could be used, e.g., glass, plastics or specific polymers, for example, TPE (thermo plastic elastomer); LSR (liquid silicone rubber); LDPE (low density polyethylene); and/or any kind of medical grade rubber, natural or synthetic. By “substantially all” it is meant that at least about 80% of the second medicament is expelled from the drug delivery device. However, it may be desirable for at least about 90% to be expelled.

The medicated module disclosed herein may be used with any drug delivery device with an appropriate compatible connection interface. However, the medicated module may be designed to limit its use to one exclusive primary drug delivery device (or family of devices) through employment of dedicated/coded/exclusive features to prevent attachment of a non-appropriate medicated module to a non-matching device. In some situations it may be beneficial to ensure that the medicated module is exclusive to one drug delivery device while also permitting the attachment of a standard drug dispense interface to the device. This would allow the user to deliver a combined therapy when the module is attached, but would also allow delivery of the primary compound independently through a standard drug dispense interface in situations, such as, but not limited to, dose splitting or top-up of the primary compound.

The medicated module disclosed herein makes it possible to tailor dose regimes when required, especially where a titration period is necessary for a particular drug. The medicated module could be supplied in a number of titration levels with obvious differentiation features such as, but not limited to, aesthetic design of features or graphics, numbering etc, so that a patient could be instructed to use the supplied medicated module in a specific order to facilitate titration. Alternatively, the prescribing physician may provide the patient with a number of “level one” titration medicated modules and then when these were finished, the physician could then prescribe the next level. The medicaments making up the combination dose may be delivered as discrete units (e.g., sequentially) or as a mixed unit (e.g., simultaneously).

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Exemplary embodiments are described herein with reference to the accompanying drawings, in which:

FIG. 1 illustrates an exemplary drug delivery device that can be used with the medicated module disclosed herein;

FIG. 2 illustrates an exemplary cartridge holder that may be used in connection with the drug delivery device of FIG. 1;

FIG. 3 illustrates an exemplary medicated module;

FIG. 4 illustrates a cross-sectional view of another exemplary medicated module;

FIG. 5a illustrates another exemplary medicated module in a priming state;

FIG. 5b illustrates a zoomed in view of various components of the medicated module in a priming state;

FIG. 5c illustrates the outer housing of the medicated module of FIG. 5a;

FIG. 5d illustrates the bypass housing of the medicated module of FIG. 5a;

FIG. 5e illustrates the needle guard of the medicated module of FIG. 5a;

FIG. 5f illustrates the medicated module of FIG. 5a where the needle guard is being pressed against the skin of a user;

FIG. 5g illustrates a zoomed in view of various components of the medicated module as the needle guard is being pressed against the skin of a user;

FIG. 5h illustrates a zoomed in view of various components of the medicated module when the medicated module is in an activated state;

FIG. 5i illustrates the medicated module of FIG. 5a when the medicated module is in a delivery state;

FIG. 5j illustrates a zoomed in view of various components of the medicated module when the medicated module is in a delivery state;

FIG. 6a illustrates another exemplary medicated module in a priming state;

FIG. 6b illustrates a zoomed in view of various components of the medicated module in a priming state;

FIG. 6c illustrates the outer housing of the medicated module of FIG. 6a;

FIG. 6d illustrates the bypass housing of the medicated module of FIG. 6a;

FIG. 6e illustrates the needle guard of the medicated module of FIG. 6a;

FIG. 6f illustrates the medicated module of FIG. 6a where the needle guard is being pressed against the skin of a user;

FIG. 6g illustrates a zoomed in view of various components of the medicated module as the needle guard is being pressed against the skin of a user;

FIG. 6h illustrates a zoomed in view of various components of the medicated module when the medicated module is in an activated state;

FIG. 6i illustrates the medicated module of FIG. 6a when the medicated module is in a delivery state; and

FIG. 6j illustrates a zoomed in view of various components of the medicated module when the medicated module is in a delivery state.

### DETAILED DESCRIPTION

The exemplary medicated modules disclosed herein allow a user to administer a user settable dose of a first medicament and a fixed dose of a second medicament (collectively “a combination dose”) through a single dispense interface, where only a single dose setting step is required, and where the medicaments and/or fluids are not combined until delivery. Further, the exemplary medicated modules disclosed herein transition from a pre-activation/priming state to a delivery state automatically upon sufficient axial refraction of the needle guard component during insertion of the dispense interface into the skin of the user.

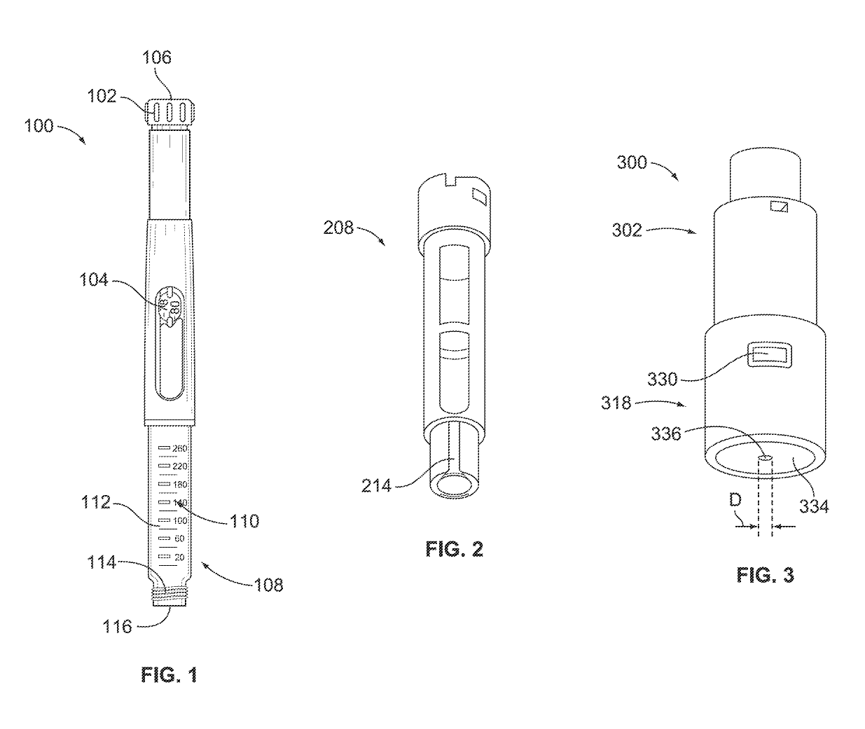

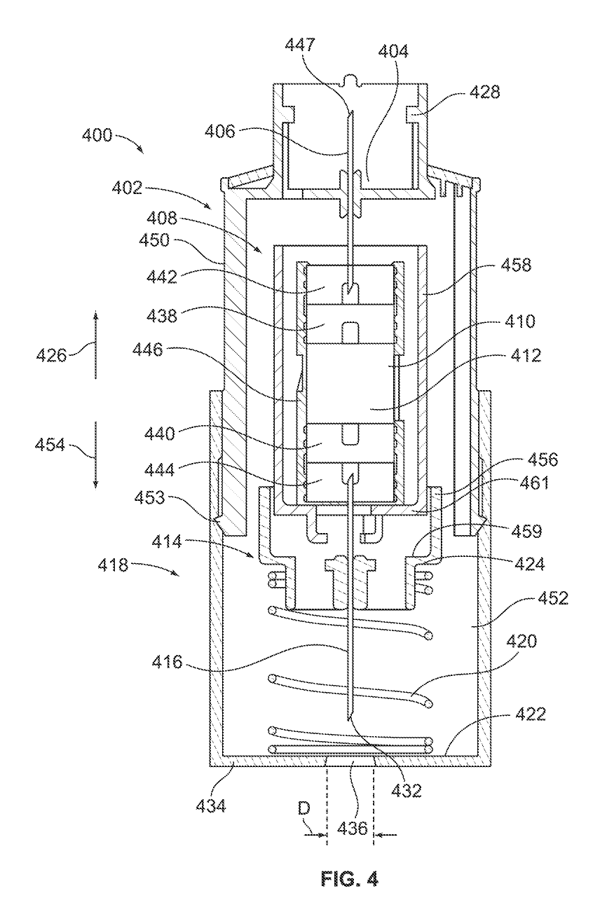

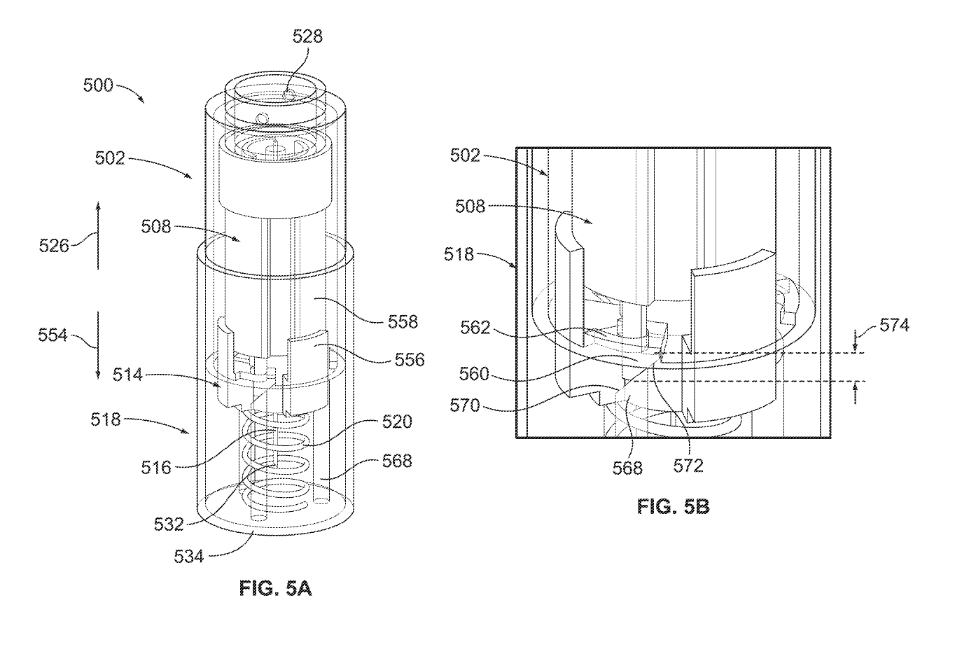

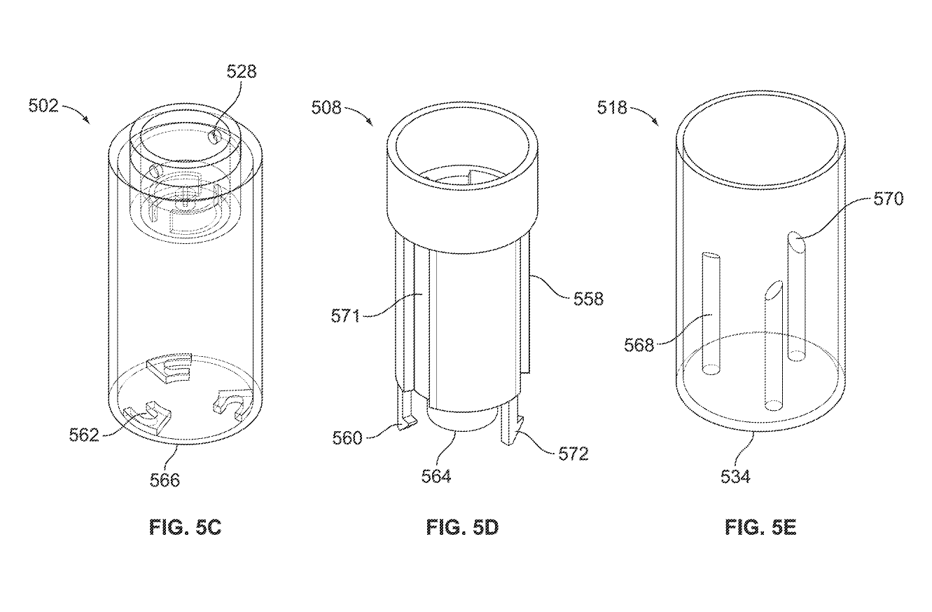

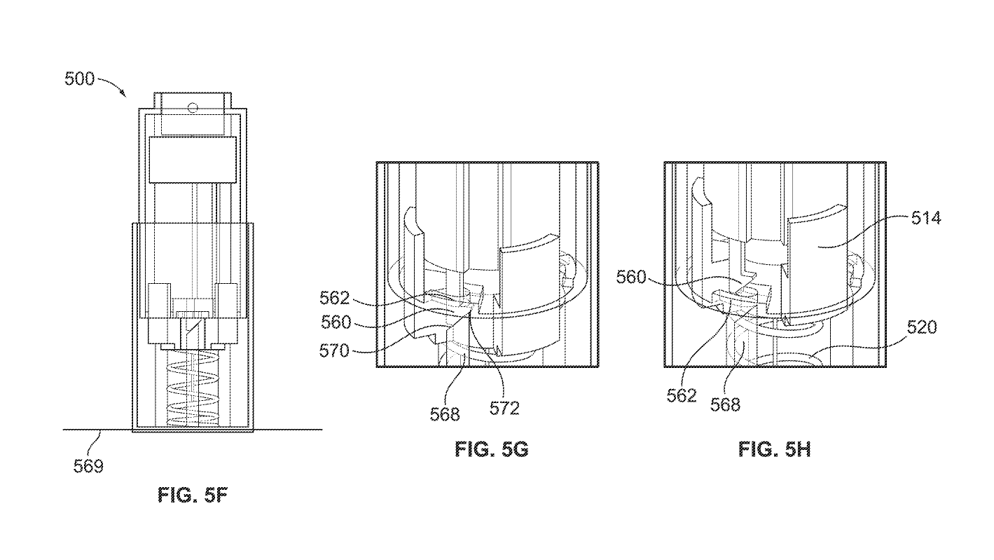

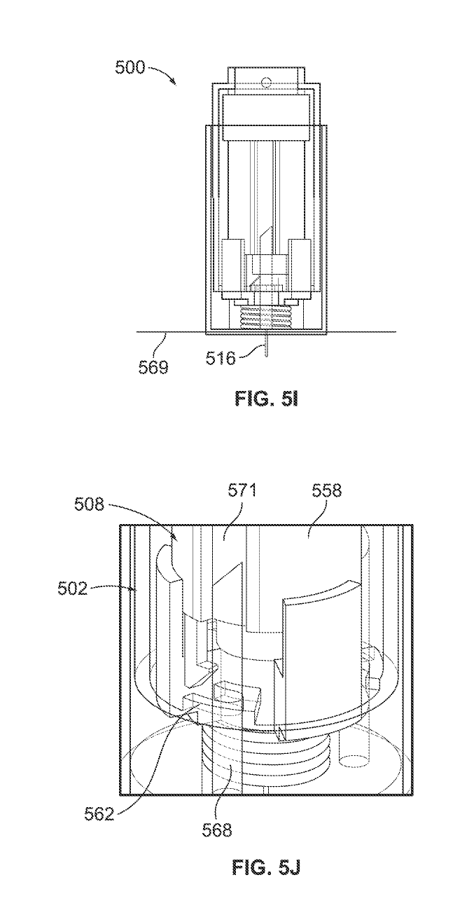

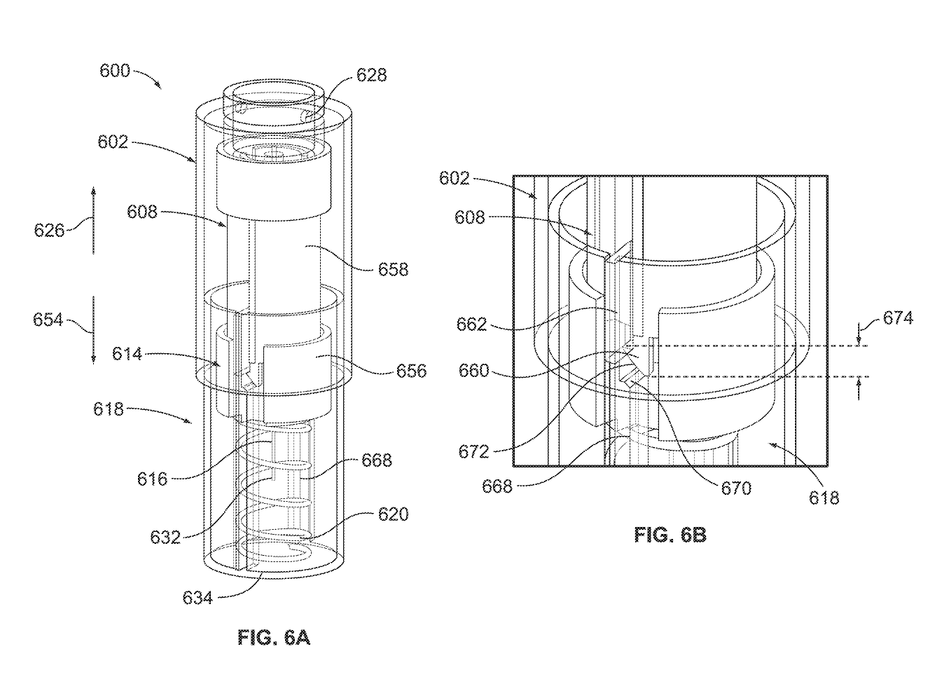

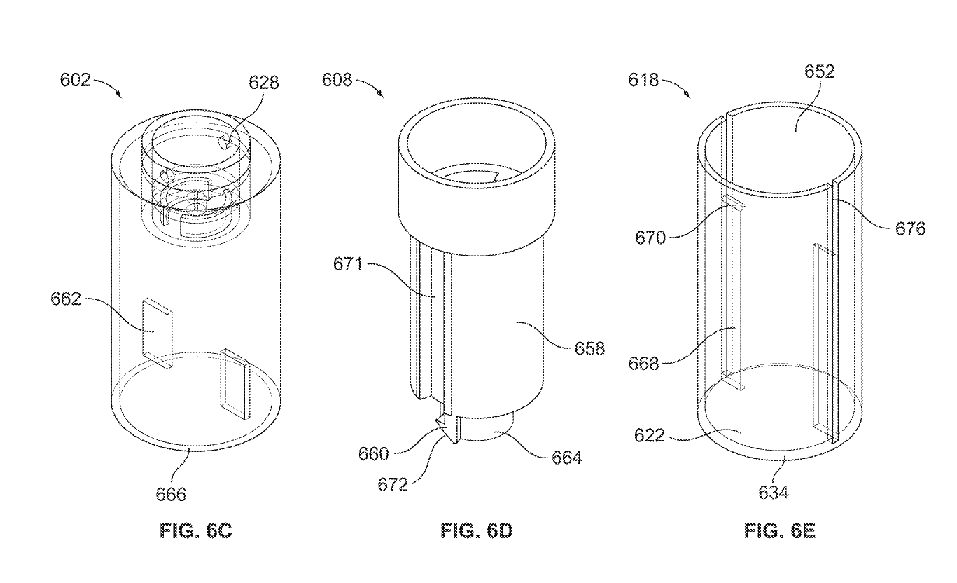

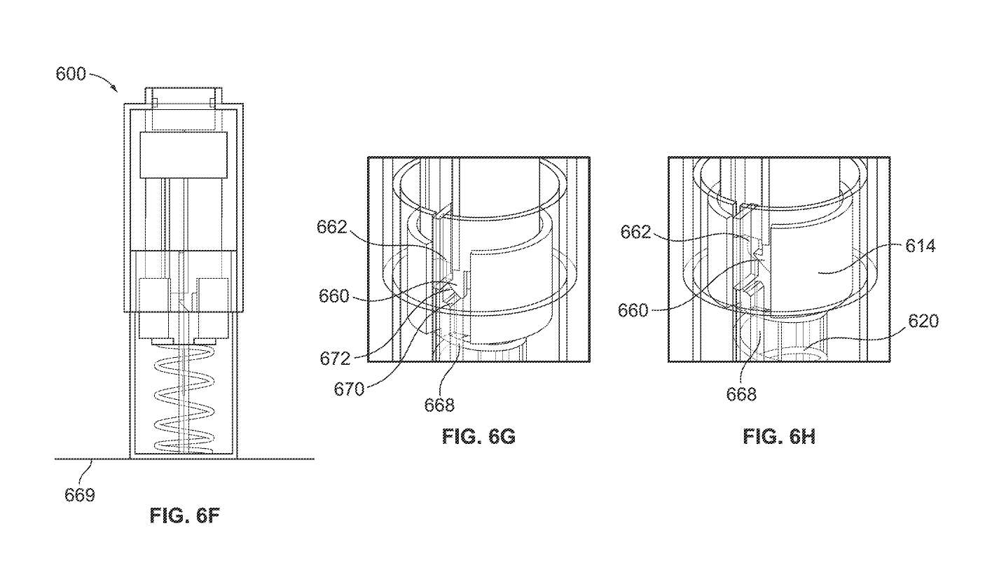

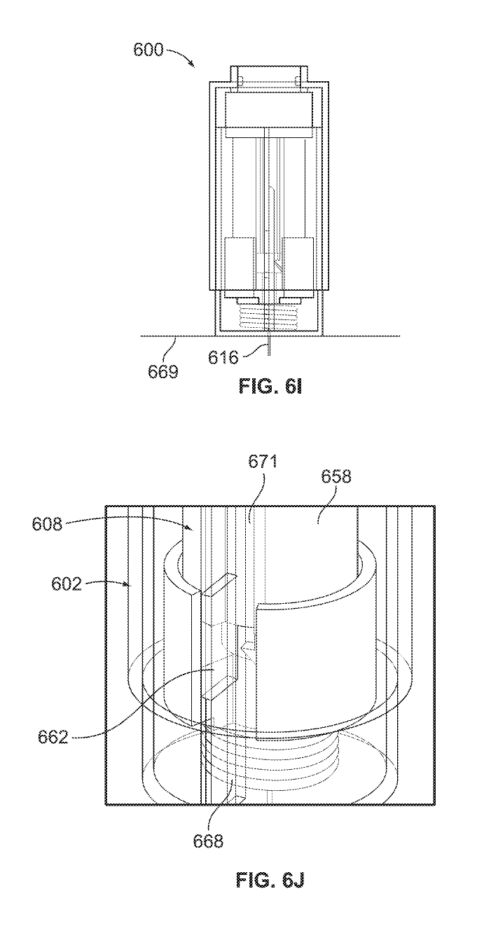

As disclosed herein, and with reference to the exemplary medicated module shown in FIG. 4, the medicated module 400 comprises: (i) an outer housing 402 configured to attach to a drug delivery device (such as drug delivery device 100 shown in FIG. 1) that contains a first medicament 112, where an upper hub 404 of the outer housing 402 holds a proximal needle 406, (ii) a bypass housing 408 including a reservoir 410 containing a second medicament 412, (iii) a lower hub 414 that holds a distal needle 416 (i.e., the dispense interface), (iv) a slidable needle guard 418, and (v) a biasing member 420 such as a spring, where the biasing member 420 is located between a proximally facing internal surface 422 of the needle guard 418 and a distally facing external surface 424 of the lower hub 426. The biasing member 420 is configured to force the lower hub 414 and the bypass housing 408 in the proximal direction 426 after the medicated module 400 is activated, thereby placing the second needle 416 in fluid communication with both the first and second medicaments 112, 412 and thus allowing the combination dose to be delivered (herein, sometimes referred to as “dispensed”). Herein, activation of the medicated module 400 refers to the disengagement of the outer housing 402 and the bypass housing 408 which results in the distal needle 416 coming into fluid communication with the first and second medicaments 112, 412 as the biasing member 420 releases stored energy. Such “disengagement” will be described in detail with reference to FIGS. 5a-6j.

FIG. 1 illustrates an exemplary drug delivery device 100 that the exemplary medicated modules described herein can be attached to. The drug delivery device 100 generally comprises a dose setting mechanism that includes a dose dial 102 and a dose indicator 104, a drive mechanism that includes a delivery button 106 for activating the drug delivery device 100, and a cartridge holder 108 that holds a cartridge 110 containing a first medicament 112. The distal end of the cartridge holder has a connection means 114 that is compatible with a connection means of the medicated module (such as connection means 428 on the upper hub 404 of the exemplary medicated module 400 shown in FIG. 4) so that the medicated module can be securely attached to the drug delivery device 100. The cartridge 110 is sealed at its distal end with a septum 116. As shown, the cartridge 110 contains multiple doses of the first medicament 112, however, in other examples, the cartridge may contain only a single dose. The user can set a desired dose of the first medicament 112 by rotating the dose dial 102 until the desired dose is displayed on the dose indicator 104. Those skilled in the art will appreciate that various other drug delivery devices known in the art may be used with the various exemplary medicated modules disclosed herein without departing from the true scope and spirit of the present invention, which is defined by the claims.

Although connection means 114 is illustrated as threads in FIG. 1, any type of connection means known in the art may be used such as a snap lock, snap fit, press fit, luer lock, bayonet-type feature, snap ring, keyed slot, ratchet mechanism, and combinations thereof, keeping in mind that it is desirable to use a connection means that prevents leakage of medicament at the connection interface. FIG. 2 shows an exemplary cartridge holder 208 where the connection means 214 is a bayonet-type feature. The connection means may provide a permanent or removable attachment. For example, the connection means may include threads and one-way ratchet teeth, thereby providing a permanent attachment. Such a connection may be useful when the drug delivery device is a single use/disposable device. On the other hand, the connection means may include threads alone, thereby allowing the user to remove the medicated module and replace it with a new one after the second medicament has been dispensed. Such a connection may be desirable when the drug delivery device is a multi-use device such as the one shown in FIG. 1.

FIG. 3 illustrates an exemplary medicated module 300, however, only a needle guard 318 and an outer housing 302 are visible, therefore reference is made to some of the features of the exemplary medicated module 400 shown in FIG. 4, where such features may be used in the exemplary medicated module 300. As shown, the needle guard 318 is provided with a window 330 through which various indicators on the outer housing 302 can be viewed. In those embodiments where the needle guard is located within the outer housing (see FIGS. 6a-j), the window may be provided in the outer housing and the indicators provided on the needle guard. These indicators may represent different states of the medicated module 300 when the medicated module 300 is attached to a drug delivery device such as drug delivery device 100 shown in FIG. 1. For instance, one indicator may represent that the medicated module 300 is in a pre-activation/priming state as shown by the exemplary of the medicated modules illustrated in FIGS. 3, 4, 5a, 5b, 6a, and 6b, another indicator may represent that the medicated module 300 is in an activated state (i.e., when fluid communication exists between a distal needle 416 of the medicated module 300 and both the first medicament 112 in the drug delivery device 100 and the second medicament 412 in the medicated module 300) as shown by the examples of the medicated module illustrated in FIGS. 5h and 6h, and a third indicator may represent that the medicated module 300 is in a delivery state (i.e., when the needle guard 318 is fully retracted) such that the distal end 432 of the distal needle 416 is exposed and inserted into the skin of the user as shown by the examples of the medicated module illustrated in FIGS. 5i, 5j, 6i, and 6j. An alternative arrangement may comprise an indicator or change in indication to show that the device has been expended or locked out. The indicators may be physical protrusions, colors, graphics, symbols, and/or text.

The distal end 334 of the guard 318 has a planar surface that provides an added measure of safety and reduces the pressure exerted by the guard 318 on the injection site during an injection. The planar surface substantially covers access to the distal needle 416, which helps to prevent inadvertent needle sticks prior to and after injection and also helps to maintain sterility of the distal needle 416. The distal end 334 of the needle guard 318 also has a pass-through hole 336. The diameter D of the pass-through hole 336 must be large enough for the distal needle 416 to pass through but should not be so large that the distal needle 416 can be accessed through the hole 336.

FIG. 4 illustrates a cross-sectional view of an exemplary medicated module 400 in a pre-activation/priming state. As shown, the second medicament 412 is contained within a reservoir 410 that is sealed on both ends with a respective septum 438, 440, each of which is fixed in place using a respective keeper or plug 442, 444. The septa 438, 440 provide a hermetically sealed and sterile reservoir 410 for the second medicament 412. Each keeper 442, 444 has at least one fluid channel (not shown) that is in fluid communication with the proximal or distal needle 406, 416, and with a bypass channel 446 or channels, which may be part of the inside surface of the bypass housing 408 or may be provided by a capsule that holds the septa 438, 440, keepers 442, 444, and reservoir 410. Together, the fluid channels within the keepers 442, 444 and the bypass channel 446 create a fluid path that allows for priming of a drug delivery device, such as drug delivery device 100 shown in FIG. 1, after it has been attached to the medicated module 400.

When the medicated module 400 is attached to the drug delivery device 100, the proximal end 447 of the proximal needle 406, which is attached to the upper hub 404 of the outer housing, penetrates the septum 116 sealing the distal end of the cartridge 110 containing the first medicament 112, thereby placing the proximal and distal needles 406, 416 in fluid communication with the first medicament 112. In other words, the first medicament is capable of flowing through the both needles 406, 416. At this point, the system can be primed by dialing out and dispensing a small number of units (or cocking the drug delivery device if only a single dose selection is possible) of the first medicament 112 using the dose dial 102 and delivery button 106 of the drug delivery device 100. During priming, the first medicament 112 flows through the proximal needle 406, into and through the fluid channel of the proximal keeper 442, through the bypass channel 446, into and through the fluid channel of the distal keeper 444, and finally out through distal needle 416.

In this example, the needle guard 418 is slidably engaged with the outer surface 450 of the outer housing 402. However, as noted above, the examples shown in FIGS. 6a-j include a guard that is slidably engaged with an inner surface of the outer housing. This engagement may be accomplished using a pin and channel arrangement (not shown), where one or more pins may be located on the inner surface 452 of the guard 418 and one or more corresponding channels may be located on the outer surface 450 of the outer housing 402 or vice versa. These channels should be configured to allow the guard 418 to slide in the longitudinal direction (i.e., proximally and/or distally) without rotating. In other examples engagement of the needle guard 418 and the outer surface 450 may be accomplished using a different arrangement such as a fin and channel arrangement. The outer housing 402 and/or guard 418 may include retention features 453 to prevent the guard 418 from disengaging the outer housing 402 at its fully extended position, as shown in FIG. 4.

The spring 420 is positioned between the proximally facing internal surface 422 of the guard 418 and a distally facing external surface 424 of the lower hub 414. Upon assembly, the spring 420 is partially compressed to supply a proximally directed 426 biasing force against the lower hub 414 and a distally directed 454 biasing force against the needle guard 418. This pre-compression of the spring 420 is possible without causing either the proximal or distal needle 406, 416 to pierce their respective septa 438, 440 because the lower hub 414 and the bypass housing 408 are prevented from moving in the proximal direction 426 until activation of the medicated module 400, while the needle guard 418 is prevented from moving in the distal direction 454 past its fully extended position. The bypass housing 408 is prevented from moving in the proximal direction 426 relative to the outer housing 402 by compatible engagement features described below in great detail with reference to FIGS. 5a-6j on the bypass housing 408 and the outer housing 402 while the lower hub 414 is prevented from moving in the proximal direction 426 relative to the bypass housing 408 by engagement of the legs 456 of the lower hub 414 and respective stand off features on the outer surface 458 of the bypass housing 408. As noted above, the retention features 453 of the needle guard 418 and/or outer housing 402 prevent the guard 418 from moving in the distal direction 454 past its fully extended position.

FIGS. 5a-j show an exemplary medicated module 500 where the compatible engagement features 560, 562 of the bypass housing 508 and the outer housing 502 are illustrated in detail. Because some of the features of the medicated module 500 are not visible in FIGS. 5a-j, reference will be made to features of the exemplary medicated module 400 shown in FIG. 4, where such features may be used with the exemplary medicated module 500. Further, although not shown attached to a drug delivery device, a drug delivery device such as device 100 shown in FIG. 1 may be attached to the medicated module 500.

As shown best in FIG. 5b, the bypass housing 508 has three hook engagement features 560 near its distal end 564 (see FIG. 5d) that engage three compatible cutout or slot engagement features 562 near the distal end 566 (see FIG. 5c) of the outer housing 502 when the medicated module 500 is in a pre-activation/priming state. Although three hook and cutout engagement features 560, 562 are shown, it should be understood that any number of hook and compatible cutout engagement features 560, 562 may be used.

To activate the medicated module 500, the user presses the distal surface 534 of the needle guard 518 against their skin 569 (see FIG. 5f) until the disengagement members/posts 568 of the needle guard 518 cause the hook and cutout engagement features 560, 562 to disengage as shown in FIG. 5h. As the user presses the distal surface 534 of the needle guard 518 against their skin the angled surfaces 570 of the posts 568 interact with the angled surfaces 572 of the hook engagement features 560 as shown best in FIG. 5g. This interaction causes the bypass housing 508 to rotate. This eventually leads to disengagement of the hook and cutout engagement features 560, 562 (see FIG. 5h) at a pre-defined proximal displacement (equal to approximately the distance 574 shown in FIG. 5b) of the needle guard 518. In addition, the rotation of the bypass housing 508 also disengages the legs 556 of the lower hub 514 from stand off features (not shown) on the outer surface 558 of the bypass housing 508, thus allowing the lower hub 514 to move proximally 526 with respect to the bypass housing 508. The lower hub is keyed to the outer housing to stop it from rotating but allowing axial movement under the action of the spring.

Upon disengagement of (i) the bypass housing 508 and the outer housing 502 and (ii) the legs 556 of the lower hub 514 and the stand-off features of the bypass housing 508, the spring 520 releases stored energy which forces the lower hub 514 and the bypass housing 508 in the proximal direction 526 thereby causing the distal needle 516 to pierce the distal septum 440 and the proximal needle 406 to pierce the proximal septum 438. More specifically, upon disengagement, the lower hub 514 moves in the proximal direction 526 relative to the bypass housing 508, which causes the proximal end of the distal needle 516 to pierce the distal septum 440. When the lower hub 514 can no longer move in the proximal direction 526 relative to the bypass housing 508 (because of the interaction between surfaces and/or features of the lower hub 514 and bypass housing 508, such as the surface 459 of the lower hub 514 and the surface 461 of the bypass housing 508), the force being applied to the lower hub 514 by the spring 520 forces the lower hub 514 and the bypass housing 508 to move together in the proximal direction 526, which causes the distal end of the proximal needle 406 (which is not moving) to pierce the proximal seal 438. The medicated module 500 is now in an activated state as the distal needle 516 is in fluid communication with both a first medicament in a drug delivery device and the second medicament 412 in the medicated module 500. In other words, if a drug delivery device, such as drug delivery device 100 shown in FIG. 1, were attached to the medicated module 500 in its activated state and the drug delivery device 100 were activated (e.g., by actuating delivery button 106), the first medicament 112 would flow through the reservoir 410 in the medicated module 500 and out of the distal needle 516. This would force the second medicament 412 out of the reservoir 410 and through the distal needle 516 as well. However, the medicated module 500 is not yet in its ready-to-use/delivery state because the distal end 532 of the distal needle 516 has not yet pierced the skin 569 of the user.

After the medicated module 500 is activated, the user continues to press the needle guard 518 against their skin 569 until the guard 518 is completely retracted (i.e., it cannot move further in the proximal direction 526) and the distal needle 516 has pierced the user's skin 569 as shown in FIGS. 5i and 5j. At this point, the medicated module 500 is in a delivery state and both medicaments 112, 412 can be subcutaneously delivered to the user upon actuation of the delivery button 106 of the drug delivery device 100. As shown in FIG. 5j, the cutout engagement features 562 of the outer housing 502 are configured to allow the disengagement members/posts 568 of the needle guard 518 to pass through when the needle guard 518 is displaced in the proximal direction 526 after activation of the medicated module 500. Further, as shown, the outer surface 558 of the bypass housing 508 has grooves/recesses 571 configured to accommodate the disengagement members/post 568 as they move in the proximal direction 526. After the user has administered a combination dose of the medicaments 112, 412 and is no longer applying a distally directed 554 force to the drug delivery device/medicated module, the needle guard 518 will return to its fully extended position. This helps ensure user safety. Although not shown, the needle guard 518 and/or outer housing 502 may include features that lock the needle guard 518 in its fully extended position after use.

FIGS. 6a-j show another exemplary medicated module 600 where the engagement feature 662 on the outer housing 602 and the disengagement member 668 of the needle guard 618 are different than those of the exemplary medicated module 500 shown in FIGS. 5a-j. Further, the needle guard 618 is positioned such that its outer surface slides against the inner surface of the outer housing 602. However, the functionality/operation of the exemplary medicated module 600 shown in FIGS. 6a-j is similar to that of the medicated module 500 shown in FIGS. 5a-j. Again, because some of the features of the medicated module 600 are not visible in FIGS. 6a-j, reference will be made to features of the exemplary medicated module 400 shown in FIG. 4, where such features may be used with the exemplary medicated module 600. Further, although not shown attached to a drug delivery device, a drug delivery device such as device 100 shown in FIG. 1 may be attached to the medicated module 600.

As shown best in FIG. 6b, the bypass housing 608 has three hook engagement features 660 near its distal end 664 (see FIG. 6d) that engage three compatible rib features 662 near the distal end 666 (see FIG. 6c) of the outer housing 602 when the medicated module 600 is in a pre-activation/priming state. Although three hook and rib engagement features 660, 662 are shown, it should be understood that any number of hook and compatible rib engagement features 660, 662 may be used.

To activate the medicated module 600, the user presses the distal surface 634 of the needle guard 618 against their skin 669 (see FIG. 6f) until disengagement members 668 of the needle guard 618 cause the hook and rib engagement features 660, 662 to disengage as shown in FIG. 6h. As shown, the disengagement members 668 are not posts as in exemplary medicated module 500, rather they are ribs or fins that protrude from the proximally facing surface 622 of the guard 618 and/or from the inner surface 652 of the guard 618. As the user presses the distal surface 634 of the needle guard 618 against their skin the angled surfaces 670 of the disengagement members 668 interact with the angled surfaces 672 of the hook engagement features 660 as shown best in FIG. 6g. This interaction causes the bypass housing 608 to rotate which eventually leads to disengagement of the hook and rib engagement features 660, 662 (see FIG. 6h) at a pre-defined proximal displacement (equal to the approximately the distance 674) of the needle guard 618. In addition, the rotation of the bypass housing 608 also disengages the legs 656 of the lower hub 614 from stand off features (not shown) on the outer surface 658 of the bypass housing 608, thus allowing the lower hub 614 to move proximally 626 with respect to the bypass housing 608.

Upon disengagement of (i) the bypass housing 608 and the outer housing 602 and (ii) the legs 656 of the lower hub 614 and the stand-off features of the bypass housing 608, the spring 620 releases stored energy which forces the lower hub 614 and the bypass housing 608 in the proximal direction 626 thereby causing the distal needle 616 to pierce the distal septum 440 and the proximal needle 406 to pierce the proximal septum 438. More specifically, upon disengagement, the lower hub 614 first moves in the proximal direction 626 relative to the bypass housing 608, which causes the proximal end of the distal needle 616 to pierce the distal septum 440. When the lower hub 614 can no longer move in the proximal direction 626 relative to the bypass housing 608 (because of the interaction between surfaces and/or features of the lower hub 614 and bypass housing 608, such as the surface 459 of the lower hub 614 and the surface 461 of the bypass housing 608), the force being applied to the lower hub 614 by the spring 620 forces the lower hub 614 and the bypass housing 608 to move together in the proximal direction 626, which causes the distal end of the proximal needle 406 (which is not moving) to pierce the proximal seal 438. The medicated module 600 is now in an activated state as the distal needle 616 is in fluid communication with both a first medicament in a drug delivery device and the second medicament 412 in the medicated module 600. In other words, if a drug delivery device, such as drug delivery device 100 shown in FIG. 1, were attached to the medicated module 600 in its activated state and the drug delivery device 100 were activated (e.g., by actuating delivery button 106), the first medicament 112 would flow through the reservoir 410 in the medicated module 600 and out of the distal needle 616, thereby forcing the second medicament 412 out of the reservoir 410 and through the distal needle 616 as well. However, the medicated module 600 is not yet in its ready-to-use/delivery state because the distal end 632 of the distal needle 616 has not yet pierced the skin 669 of the user.

After the medicated module 600 is activated, the user continues to press the needle guard 618 against their skin 669 until the guard 618 is completely retracted (i.e., it cannot move further in the proximal direction 626) and the distal needle 616 has pierced the user's skin as shown in FIGS. 6i and 6j. At this point the medicated module 600 is in a delivery state and both medicaments 112, 412 can be subcutaneously delivered to the user upon actuation of the delivery button 106 of the drug delivery device 100. As shown best in FIG. 6j, after activation of the medicated module 600, the disengagement members 668 are able to slide past the rib engagement features 662 when the needle guard 618 is displaced in the proximal direction 626 after activation of the medicated module 600. This is possible because of the slot features 676 in the needle guard 618 which are offset from the disengagement members 668. These slot features 676 act as respective tracks for each of the rib engagement features 662. These slot features 676 also help to accommodate the needle guard 618 being positioned within the outer housing 602. Further, as shown, the outer surface 658 of the bypass housing 608 has grooves/recesses 671 configured to accommodate the disengagement members 668 as they move in the proximal direction 526. After the user has administered a combination dose of the medicaments 112, 412 and is no longer applying a distally directed 654 force to the drug delivery device/medicated module, the needle guard 618 will return to its fully extended position. This helps to ensure user safety. Although not shown, the needle guard 618 and/or outer housing 602 may include features (e.g., a biasing spring) that lock the needle guard 618 in its fully extended position after use.

To help minimize the residual volume of the second medicament that might remain in the reservoir of the medicated module at the end of the dispense operation due to recirculation and/or stagnant zones, a flow distributor may be provided as an integral part of the reservoir. The design of flow distributor could ensure that at least about 80% of the second medicament is expelled from the reservoir through the distal end of the dispense interface when the first medicament is forced through the reservoir. In some cases it may be desirable to ensure that at least about 90% is expelled. In cases where plug flow is achieved, displacement of the first medicament from the drug delivery device and through the reservoir of the medicated module will displace the dose of the second medicament stored in the reservoir without substantial mixing of the two medicaments. A flow distributor may also help to minimize diffusion of the second medicament into the first medicament during delivery.

The reservoir and flow distributor may be manufactured as a single part from materials that are compatible with the first and second medicaments, perhaps as a single molded piece. The flow distributor may be configured and positioned in the reservoir such that the second medicament fills flow channels that are defined by the shape and location of one or more channels inside the reservoir. The shape of the flow channels can be optimized for a plug flow of medicament by varying the dimensions of the flow distributor and/or channels. The cross-sectional area of the annulus formed between the flow distributor and the wall of the reservoir should be kept relatively small. The volume available to store the secondary medicament would equal the internal volume of the reservoir minus the volume of the flow distributor. Therefore if the volume of the flow distributor is marginally smaller than the internal volume of the capsule, a small volume is left which the secondary medicament occupies.

Exemplary embodiments of the present invention have been described. Those skilled in the art will understand, however, that changes and modifications may be made to these embodiments without departing from the true scope and spirit of the present invention, which is defined by the claims.

1-14. (canceled)

15. A medicated module configured for use with a drug delivery device containing a first medicament, the medicated module comprising:
an outer housing including an upper hub located near a proximal end of the outer housing, wherein the upper hub holds a first needle, and wherein the proximal end is configured to attach to the drug delivery device;
a bypass housing including a reservoir containing a second medicament, wherein the bypass housing is configured to be engaged with the outer housing prior to activation of the medicated module;
wherein prior to activation the bypass housing is prevented from moving in the proximal direction relative to the outer housing;
a lower hub including a second needle;
a slidable needle guard including at least one disengagement member, wherein the needle guard is configured to activate the medicated module by forcing the bypass housing to disengage from the outer housing at a pre-defined amount of proximal displacement of the needle guard; and
a biasing member located between a proximally facing internal surface of the needle guard and a distally facing external surface of the lower hub,
wherein the biasing member is configured to force the lower hub and the bypass housing in the proximal direction after activation of the medicated module, thereby placing the second needle in fluid communication with the first and second medicaments.

16. The medicated module of claim 15, wherein the biasing member comprises a spring.

17. The medicated module claim 15, wherein the outer housing further includes an opening for viewing indicia on an outer surface of the needle guard, where the indicia indicates one or more of a priming state, an activated state, a ready to deliver and a lock-out state.

18. The medicated module of claim 15, wherein the needle guard (318; 418;
618) is rotationally constrained by the outer housing.

19. The medicated module of claim 15, wherein the bypass housing further includes a bypass channel configured to allow a priming dose of the first medicament to bypass the reservoir containing the second medicament.

20. The medicated module of claim 15, wherein each of the first and second needles comprises a double-ended needle.

21. The medicated module of claim 15, wherein the bypass housing further includes at least one first engagement feature, wherein the outer housing further includes at least one second engagement feature, and wherein the first and second engagement features are configured to be engaged prior to activation of the medicated module.

22. The medicated module of claim 21, wherein the at least one first engagement feature comprises at least one hook engagement feature.

23. The medicated module of claim 22, wherein the at least one second engagement feature comprises at least one of at least one cutout engagement feature and at least one rib protrusion feature.

24. The medicated module of claim 23, wherein the at least one second engagement feature is configured to allow the at least one disengagement member of the needle guard to pass through when the needle guard is displaced in the proximal direction after activation of the medicated module.

25. The medicated module of claim 15, wherein a proximal surface of the at least one disengagement member and a distal surface of the at least one first engagement feature are each angled such that proximal displacement of the at least one disengagement member forces the bypass housing to rotate due to interaction of the angled proximal surface of the at least one disengagement member and the angled distal surface of the at least one first engagement feature, and wherein the bypass housing continues to rotate until the first and second engagement features are disengaged.

26. The medicated module of claim 15, wherein an outer surface of the bypass housing includes at least one recess configured to prevent interference with the at least one disengagement member when the needle guard is displaced in the proximal direction after activation of the medicated module.

27. The medicated module of claim 15, wherein the at least one disengagement member protrudes from the proximally facing internal surface of the needle guard.

28. The medicated module of claim 15, wherein the at least one disengagement member comprises at least one of a post and a fin.
